(19) United States
(12) Patent Application Publication
(10) Pub. No.: US 2002/0193335 A1
(43) Pub. Date: Dec. 19, 2002

(54) Gene therapy for neurological tissues

(76) Inventors: David P. Hesson, Malvern, PA (US); Glen D. Frazer, Wynnewood, PA (US); Bruce Shook, Devon, PA (US)

Correspondence Address: ALLEN BLOOM, C/O DECHERT, PRINCETON PIKE CORPORATION CENTER, P.O. BOX 5218, PRINCETON, NJ, 08543-5218, US

(21) Appl. No.: 10/090,445

(22) Filed: Mar. 4, 2002

Related U.S. Application Data
(60) Provisional application No. 60/327,570, filed on Mar. 2, 2001.

(51) Int. Cl. A61K 48/00

(52) U.S. Cl. 514/44; 424/93.21

(57) ABSTRACT

Provided is a method of delivering to neurological tissue a transforming composition or a transformed cell, the method comprising: a. injecting a physiologically acceptable cerebrospinal perfusion fluid into a first catheter into the cerebrospinal pathway, which cerebrospinal perfusion fluid has a gene therapy effective amount of gene therapy agent or transformed cells; b. withdrawing fluid at a second catheter into the cerebrospinal pathway to create a flow and flow pathway between the first and second catheters; and c. maintaining the flow for a period of time adapted to perfuse at least 1 CSF volume.

This Application claims the priority of U.S. Provisional Application No. 60/______ (pursuant to a petition converting U.S. patent application Ser. No. 09/798,775 to a provisional), which application was filed Mar. 2, 2001.

This invention relates to gene therapy formulations or compositions and methods useful for conducting gene therapy for neurological tissue, of the central nervous system (CNS). The compositions can contain an artificial cerebrospinal fluid (ACSF) carrier component and can further contain nutrients.

The cerebrospinal fluid (CSF) pathway system, which intimately bathes and permeates brain and spinal cord tissues, constitutes a circulatory system within the body. Although it has some similarities to systemic vascular and lymphatic circulation, its anatomical arrangement differs considerably. Indeed, this system has been named the “third circulation” system. Due to the extensive area of CSF-tissue contact over the cerebral and spinal cord surfaces, in the paravascular Virchow-Robins spaces, and cerebral ventricles, the cerebrospinal fluid system constitutes a vast, complex and intimate avenue for access to central nervous tissue.

In gene therapy, one typically seeks to transfect or transform cells of a certain cell type, such as liver cells, pancreatic cells, lung cells, muscle cells, leukocytes and the like, to insert an gene to correct a genetic defect, express a therapeutic protein, or otherwise provide a helpful function. Such a gene can include a nucleic acid construct that expresses an antisense RNA to interfere in the expression of a certain mRNA or one or more constructs that express two complementary strands designed to interfere in the expression of a certain mRNA. Similarly, nucleic acid-based vaccines seek to induce a percentage of cells to produce immune-reaction inducing polypeptides, to induce an antibody-based or cellular-based immune response. Also, one can seek to inhibit the function of genes or gene products with antisense molecules.

The present invention provides efficient avenues for delivering gene therapy agents to neuronal tissue.

### SUMMARY OF THE INVENTION

In one embodiment, the invention provides a method of delivering to neurological tissue of the CNS a transforming composition or a transformed cell, the method comprising: a. injecting a physiologically acceptable cerebrospinal perfusion fluid (CSPF) into a first catheter into the cerebrospinal pathway, which CSPF has a gene therapy effective amount of gene therapy agent or transformed cells; b. withdrawing fluid at a second catheter into the cerebrospinal pathway to create a flow and flow pathway between the first and second catheters; and c. maintaining the flow for a period of time adapted to perfuse at least 1 CSF volume.

In another embodiment, the invention provides a method of delivering to neurological tissue a transforming composition or a transformed cell, the method comprising: a. injecting a physiologically acceptable cerebrospinal flushing fluid into a first catheter into the cerebrospinal pathway, which cerebrospinal flushing fluid has an effective amount a gene therapy agent effective to transform neural cells to express calbindin, bal-2 leptin superoxide dismutase, a glial-derived neurotrophic growth factor which is NGF, BDNF or NT3, epidermal growth factor, a fibroblast growth factor which is FGF 1-9, insulin-like growth factor- 1, a platelet-derived growth factor which is PDGF-A, B or C, vascular endothelial growth factor, or ciliary neurotrophic factor; or transformed cells; b. withdrawing fluid at a second catheter into the cerebrospinal pathway to create a flow and flow pathway between the first and second catheters; and c. maintaining the flow for a period of time adapted to flush at least 1 CSF volume.

In further embodiment, the invention provides a method of delivering to neurological tissue a transforming composition or a transformed cell, the method comprising: a. injecting a cerebrospinal perfusion fluid into a first catheter into the cerebrospinal pathway, which cerebrospinal perfusion fluid has a gene therapy effective amount of transforming nucleic acid, wherein the cerebrospinal flushing fluid further comprises an emulsion-forming effective amount of a lipid composition comprised of lipids found in biological membranes; b. withdrawing fluid at a second catheter into the cerebrospinal pathway to create a flow and flow pathway between the first and second catheters; and c. maintaining the flow for a period of time adapted to flush at least 1 CSF volume.

### BRIEF DESCRIPTION OF THE DRAWINGS

FIG. 1 illustrates a perfusion pathway.

### DETAILED DESCRIPTION OF THE INVENTION

Transformation-Mediated Gene Therapy

A lesson of the last 20 plus years in which scientists have begun actively considering methods to introduce genetic material into appropriate target tissues to overcome a genetic disease has been that the effort is more complex than was initially anticipated. Some of the goals that were needed to be met to create successful gene therapy tools included: (1) efficient transduction of the target cells; (2) long-term expression of the gene; (3) lack of a disabling immune response to the vector or transduced cell; and (4) absence of toxicity. (See, Samulski et al., “Adeno-associated Viral Vectors” in Development of Human Gene Therapy, Cold Spring Harbor Laboratory Press, 1998, pp. 131-172.) All the above listed goals, especially the first three, identify areas that have given rise to barriers to efficient gene therapy. Vectors typically transduce only a percentage of the cells to which they are applied. The transducing gene is often maintained on an episome and is therefore often not a stably incorporated and maintained genetic element. Moreover, incorporation into the chromosomal DNA is often dependent on cell division, thereby limiting the scope of target tissues to replicating tissues. Viral vectors can encode proteins that induce immunity, thereby carrying the seeds for the destruction of the transduced cells. Certain viral vectors overcome some of these problems but otherwise create at least an implication of danger. For example, non-replicating forms of the human immunodeficiency virus are being engineered for use as gene therapy vectors that allow for the incorporation of the genetic material into genomic DNA. Such vectors must maintain the genetic tools by which to facilitate genomic incorporation, but must lack enough of the gene products that create infectivity, such that in this case for HIV there is no chance that recombination events will regenerate an infective particle (See, Naldini et al., “Lenti Viral Vectors” in Development of Human Gene Therapy, Cold Spring Harbor Laboratory Press, 1998, pp. 47-60).

These problems are, fortunately, now well-recognized, and the viral vectors used in gene therapy have improved to address such problems. Moreover, gene therapy can be conducted without viral vectors. Also, in other genetic transformations the problems of toxicity and immune response do not come to fore to the same degree. In nucleic acid-based vaccines, for example, an immune response is desirable, as can be a process by which expression of the transforming gene attenuates so that production of the immuno-stimulants attenuates over time.

Viral vectors have also been subject to engineering to change their target cell preference, for instance by binding or incorporating antibodies. For instance, Valsesia-Wittmann et al. modified the cell-surface binding characteristics of avian leucosis virus (J. Virol. 68: 4609-4619, 1994). Erythropoietin, which of course binds its cognate receptor, has been incorporated into Moloney murine leukemia virus (Mo-MLV) (Kasahara et al., Science 266: 1373-1376, 1994). A tumor-targeting single-chain antibody has been incorporated into spleen necrosis virus (Chu and Domburg, J. Virol. 69: 2659-2663, 1995). HIV envelope protein has been incorporated into murine leukemia viral vectors (Mammamo et al., J. Virol. 71: 3341-3345, 1997). Such targeting methods with respect to adenoviral vectors are reviewed by Reynolds and Curiel (“Strategies to Adapt Adenoviral Vectors for Gene Therapy Applications: Targeting and Integration,” in Development of Human Gene Therapy, Cold Spring Harbor Laboratory Press, 1998, pp. 111-130). Another approach with adenoviral vectors is to make hybrid vectors using the viral coat protein from an adenovirus strain with an appropriate target specificity. For example, a vector from Cobra Therapeutics is based on the Ad5 strain, but uses the coat protein from Ad35 to obtain a vector effective with dendritic cells.

As reviewed in Development of Human Gene Therapy (Cold Spring Harbor Laboratory Press, 1998), a wide variety of viral vectors have been selected or engineered for gene therapy. Moreover, nucleic acid can also be delivered successfully without the use of viral vectors.

Many but not all of the examples of compositions that have been used for non-virally mediated transfection will prove more appropriate for use to transform cells, which cells can be introduced into neurological tissues by the methods of the invention. Such methods include, for example, an early-developed method for increasing transfection efficiency was to use calcium phosphate-precipitated nucleic acid. The transfection potential of nucleic acid is increased by compacting it with polycationic polymers such as DEAE dextran (Veheri et al., Virology 27: 434-436, 1965), polylysine (Wu et al., J. Biol. Chem. 266: 14338-14342 1991), cationic peptides (Wadhwa et al., Bioconjugate Chem. 8: 81-88, 1997; and Niidome et al., J. Biol Chem. 272: 15307-15312, 1997), polyethyleneimine (Boussiff et al., Proc. Natl. Acad. Sci USA 92: 7297-7301, 1995), a glucaramide-based polyamino polymer (Goldman et al., Nat. Biotechnol. 15: 462-466, 1997), and polyamidoamine dendrimers (Dielinska et al., Biochim. Biophys. Acta 1353: 180-190, 1997). Other polymers useful as enhancers of nucleic acid uptake include erodable microspheres (Mathiowitz et al., Nature 386: 410-412, 1997) and polyvinyl pyrrolidone (Mumper et al., Pharm. Res. 13: 701-709, 1996). Other enhancers include cationic liposomes into which the nucleic acid is incorporated (Felgner et al., Proc. Natl. Acad. Sci. USA. 84:7413-7, 1987; Felgner and Ringold, Nature 337:387-8, 1989). Such liposomes, or “lipoplexes,” are believed to insert the nucleic acid into a target cell by a membrane fusion mechanism. Illustrative of the many cationic lipid formulations now available (see, Felgner et al., “Synthetic Delivery Systems,” in Development of Human Gene Therapy, Cold Spring Harbor Laboratory Press, 1998, pp. 241-260), is DOTMA (N[1-(2,3-dioleyloxy)propyl]-N,N,N-trimethylammonium). Other such cationic lipid formulations include Lipofectin™, a 1:1 (w/w) liposome formulation of the cationic lipid N-[1-(2,3-dioleyloxy)propyl]-N,N,N-trimethylammonium chloride (DOTMA) and dioleoyl phosphatidylethanolamine (DOPE), LipofectAMINE™, a 3:1 (w/w) liposome formulation of the polycationic lipid 2,3-dioleyloxy-N-[2(spermine-carboxamido)ethyl]-N,N-dimethyl-1-propanaminiumtrifluoroacetate (DOSPA) and the neutral lipid dioleoyl phosphatidylethanolamine (DOPE) in membrane-filtered water, and LipofectACE™, a 1:2.5 (w/w) liposome formulation of the cationic lipid dimethyl dioctadecylammonium bromide (DDAB) and dioleoyl phosphatidylethanolamine (DOPE) in membrane-filtered water (all from Life Technologies, Rockville, Md.).

Non-viral techniques that are more preferred for direct use in neurological tissue include erodible microspheres and polyvinyl pyrrolidone mediated techniques, as well as techniques that utilize liposomal compositions enriched in natural cationic lipids. Also preferred are gene transfers effected without such adjuvants.

Targeting techniques can also be employed which bind or affix targeting molecules to the nucleic acid or nucleic acid complex to be used for transfection. Cotton and Wagner, “Receptor-mediated Gene Delivery Strategies,” in Development of Human Gene Therapy, Cold Spring Harbor Laboratory Press, 1998, pp. 261-277.

Neuronal tissue of the CNS is to some degree immunologically privileged. The population of immune cells present is typically lower than in other tissues. Thus, when the invention is used to provide a vaccination, in some cases vaccinations by the methods of the invention are conducted as an adjunct to a prophylactic or therapeutic vaccination made by another route of administration. For example, one can use the method of the invention to recruit stimulated cells to neuronal tissue. Gene therapy approaches seeking to induce cellular immunity, e.g., to infected or neoplastic cells, can also be conducted by the methods of the invention.

The invention further encompasses using suicide bacteria (attenuated, intracellular parasitic bacteria) to deliver nucleic acid to the cytoplasm of bacteria-ingesting cells such as macrophages. See, e.g., U.S. Pat. No. 6,143,551.

The invention also encompasses delivering cells that were transformed in vitro. Such cells can include, for example, pluripotent cells such as fetal brain cells. Further included are bone marrow cells, other primary stem cells or stem cell lines, such as F9 cells, which have recently been shown to include cells capable of differentiating to neurological cells (Mezey et al., Science 290:1779-1782, Dec. 1, 2000; Brazelton et al., Science 290:1775-1779, Dec. 1, 2000).

In one embodiment, the transfection-facilitating adjuvant is the emulsified lipid component of cerebrospinal perfusion fluid (described below).

As will be recognized by those of ordinary skill, the nucleic acid sought to be introduced into cells will often include, in addition to the portion conveying the primary genetic characteristic of interest, a portion encoding a substance that is itself, or gives rise to, a molecule that is readily detectable. This “reporter” molecule serves as a surrogate for determining or estimating success in introducing the primary genetic characteristic. Where cells in culture are being transformed, a portion of the nucleic acid can encode a substance required for the cells to survive in the face of an appropriate challenge.

The nucleic acid can be single or double-stranded, though non-virally mediated techniques that seek to express a portion of the nucleic acid will typically use double-stranded nucleic acid.

It will be recognized that certain transformation techniques, such as those using transformation-promoting carrier compositions or salts, are to be implemented with care to avoid damage to neural tissue.

The documents cited herein are incorporated by reference to the extent that they describe the vectors or transforming adjuvant compositions mentioned.

Gene Therapy Based on Antisense Molecules

Events that provide disruption of the nucleic acid function by antisense molecules (such as antisense oligonucleotides) are thought to be of two types (Cohen in Antisense molecules: Antisense Inhibitors of Gene Expression, (1989) CRC Press, Inc., Boca Raton, Fla.). In the first, hybridization arrest, an antisense molecule inhibitor binds to the target nucleic acid and thus prevents its utilization, for example by sterically hindering the binding of essential proteins, such as ribosomes, to the nucleic acid. Methyl phosphonate antisense molecules and α-anomer antisense molecules are two extensively studied antisense agents which are thought to disrupt nucleic acid function by hybridization arrest (Miller and Ts'O, Anti-Cancer Drug Design, 2:117-128, 1987). Second, the formation of a hybrid with the targeted nucleic acid renders the hybrid susceptible to cleavage, for example by intracellular RNase H. A 2′-deoxyribofuranosyl antisense molecule or antisense molecule analog hybridizes with the targeted RNA and this duplex activates the RNase H enzyme to cleave the RNA strand. Phosphorothioate antisense molecules are the most prominent example of an antisense agent that is believed to primarily operate by this second mechanism.

Antisense molecules can also be adapted to bind duplex nucleic acids to form triplex complexes in a sequence specific manner via Hoogsteen base pairing (Beal et al., Science 251:1360-1363, 1991; Young et al., Proc. Natl. Acad. Sci. 88:10023-10026, 1991). Both antisense and triple helix therapeutic strategies are typically directed towards nucleic acid sequences that are involved in or responsible for establishing or maintaining disease conditions. Target nucleic acid sequences can be, for example, found in the genomes of pathogenic organisms including bacteria, yeasts, fungi, protozoa, parasites, viruses, or may be endogenous in nature. By hybridizing to and modifying the expression of a gene important for the establishment, maintenance or elimination of a disease condition, the corresponding condition can be cured, prevented or ameliorated.

Modifications have been made to the ribose phosphate backbone of antisense molecules to increase their resistance to nucleases. These modifications include use of, for example, methyl phosphonate, phosphorothioate and phosphorodithioate linkages, as well as alkylphosphonothioate or arylphosphonothioate linkages (see, U.S. Pat. No. 5,929,226 and P. Dan Cook, “Second Generation Antisense Oligonucleotides: 2′-Modifications”, Annual Reports in Medicinal Chemistry, 33:313-325, Academic Press 1998). Still further substitutions for phosphodiester linkages between a 2′ and 5′ position of adjacent nucleosides can be —N(R)—CH₂CH₂—, —CH₂—N(R)CH₂, —CH₂CH₂N(R)—, —OCH₂CH₂—, —CH₂CH₂O—,—OCH₂S—, —SCH₂CH₂—, —S(O)CH₂CH₂—, —S(O)₂CH₂CH₂—, —CH₂S(O)₂CH₂—, —CH₂SCH₂—, —NHC(O)O—, —OC(O)NH—, —OC(O)N(CH₂)— or —O—CH₂—O—, where R is lower alkyl. See, U.S. Pat. No. 5,817,781. Further modifications include modified sugar moieties such as 2′-O-alkyl ribose. Other antisense molecule modifications include those made to modulate uptake and cellular distribution. A number of modifications that alter the nature of the internucleotide linkage have also been reported in the literature. These include non-phosphorus linkages, peptide nucleic acids (PNA's) and 2′-5′ linkages.

One synthetic 2′-modification that imparts increased nuclease resistance and a very high binding affinity to nucleotides is the 2′-methoxyethoxy (MOE, 2′—OCH₂CH₂OCH₃) side chain (Baker et al., J. Biol. Chem. 272: 11944-12000, 1997; Freier et al., Nucleic Acids Res., 25: 4429-4443, 1997). One of the immediate advantages of the MOE substitution is the improvement in binding affinity, which is greater than many similar 2′ modifications such as O-methyl, O-propyl, and O-aminopropyl (Freier and Altmann, Nucleic Acids Research, 25:4429-4443, 1997). Antisense molecules and antisense molecule analogs having 2′-O-methoxyethyl-substitutions have also been shown to be antisense inhibitors of gene expression with useful features for in vivo use (Martin, Helv. Chim. Acta 78: 486-504, 1995; Altmann et al., Chimia 50: 168-176, 1996; Altmann et al., Biochem. Soc. Trans. 24: 630-637, 1996; and Altmann et al., Nucleosides 16: 917-926, 1997). Relative to DNA-based oligonucleotides, these antisense molecules display improved RNA affinity and higher nuclease resistance. Chimeric antisense molecules with 2′-O-methoxyethyl-ribonucleoside wings and a central DNA-phosphorothioate window also have been shown to effectively reduce the growth of tumors in animal models at low doses. MOE substituted antisense molecules have shown outstanding promise as antisense agents in several disease states.

Diseases That Can Be Treated

A wide variety of disease and conditions of the brain and spinal cord can be treated with one or a combination of gene therapies. Diseases or conditions such as Parkinson's, Alzheimer's and Amyotrophic Lateral Sclerosis, stroke, traumatic brain injury (TBI) or spinal cord injury (SCI) can be treated using gene therapy to introduce stem cells or proteins such as, calbindin, bcl-2, glial-derived neurotrophic growth factor (NGF, BDNF, NT3), epidermal growth factor (EGF), fibroblast growth factor (FGF 1-9), superoxide dismutase (SOD), insulin (IGF-1), platelet-derived growth factor (PDGF-A, B &C), vascular endothelial growth factor (VEGF), and ciliary neurotrophic factor (CNTF).

Examples of the uses of these are outlined in the following Table:

| DISEASE/CONDITION | GENE THERAPY | |
|---|---|---|
| Stroke, Traumatic Brain | Stem Cells | |
| Injury, Spinal Cord Injury | Cells transformed with: | |
| | 1. | nerve growth factor (NGF), |
| | 2. | brain-derived nerve growth factor |
| | | (BDNF), |
| | 3. | epidermal growth factor (EGF), |
| | 4. | vascular epidermal growth factor |
| | | (VEGF), |
| | 5. | fibroblast growth factor (FGF), or |
| | 6. | glial cell line-derived neurotrophic |
| | | factor (GDNF). |
| Parkinson's | Stem Cells producing dopamine | |
| | Cells transformed with: | |
| | 1. | nerve growth factor (NGF), or |
| | 2. | brain-derived nerve growth factor |
| | | (BDNF). |

| | |
|---|---|
| Alzheimer's | Cells transformed that produce acetyl choline |
| | (such as by expression of choline acetyl |
| | transferase) |
| Amyotrophic Lateral | Cells transformed that produce superoxide |
| Sclerosis | dismutase (SOD) |

Cerebrospinal Perfusion Fluid

In one embodiment, the cerebrospinal perfusion fluid is an oxygen-carrying nutrient emulsion according to the following Table.

OXYGEN CARRYING FORMULATIONS:

| | | More | Still More |
|---|---|---|---|
| | Preferred | Preferred | Preferred Range |
| Component | Range | Range | or Amount |
| Oxygen-Carrying | 5-15 | 9-11 | 9.5-10-5 |
| Compound, % v/v | | | |
| Lipid, mg/mL | 8-14 | 10-13 | 11.5 |
| Albumin, g/dL, | 0.05-2.0 | 1.5-1.9 | 1.67 |
| α-Ketoglutaric Acid, | 5-40 | 22-28 | 25 |
| μg/mL | | | |
| Amino Acids, μg/mL | | | |
| L-Isoleucine + | 5-50 | 11-23 | 17.5 |
| L-Leucine | | | |
| L-Valine | 5-50 | 11-22 | 16.6 |
| L-Alanine | 5-50 | 19-38 | 28.6 |
| L-Serine | 5-50 | 16-33 | 24.6 |
| L-Histidine | 2-20 | 7-14 | 10.3 |
| L-Methionine | 0.1-5 | 1.4-2.8 | 2.1 |
| L-Phenylalanine + | 5-50 | 23-47 | 35.3 |
| L-Lysine | | | |
| L-Threonine + | 5-50 | 32-64 | 48.3 |
| L-Arginine | | | |
| L-Tyrosine | 1-20 | 5-11 | 7.9 |
| Na⁺, mM | 135-150 | 137-147 | 147 |
| K⁺, mM | 2.5-4.0 | 2.7-3.9 | 2.9 |
| Cl⁻, mM | 110-135 | 116-135 | 130 |
| Ca⁺², mM | 1.0-1.6 | 1.0-1.5 | 1.15 |
| Mg⁺², mM | 0.8-1.6 | 1.0-1.5 | 1.12 |
| Glucose (dextrose), | 10-150 | 30-100 | 94 |
| mg/dL | | | |

The pH of the emulsion, or vehicle (constituting the above or the like without oxygen-carrying compound), is in the physiological range, such as about pH 7.3. In one embodiment, the amino acids include tryptophan.

The cerebrospinal perfusion fluid is preferably formulated such that it is physiologic and can directly contact tissues of the neuraxis for an extended period of time, from hours to days, without causing side effects. For best performance, it is believed that the artificial cerebrospinal fluid should be appropriately buffered and have appropriate amounts of amino acids, electrolytes and other compounds helpful to healthy metabolism. Thus, in preferred methods, these components do not need to be supplied through equilibration with other body fluids. Of course, simpler solutions, such as appropriately balanced salts, are used in neurosurgery and are to some degree acceptable. Where the cerebrospinal perfusion fluid is formulated with nutrients, it can be termed “artificial cerebrospinal fluid” or “ACSF.”

NON-OXYGEN CARRYING FORMULATIONS:

| | | More | Still More |
|---|---|---|---|
| | Preferred | Preferred | Preferred Range |
| Component | Range | Range | or Amount |
| Albumin, g/dL, | 0.05-2.0 | 1.5-1.9 | 1.67 |
| α-Ketoglutaric Acid, | 5-40 | 22-28 | 25 |
| μg/mL | | | |
| Amino Acids, μg/mL | | | |
| L-Isoleucine + | 5-50 | 11-23 | 17.5 |
| L-Leucine | | | |
| L-Valine | 5-50 | 11-22 | 16.6 |
| L-Alanine | 5-50 | 19-38 | 28.6 |
| L-Serine | 5-50 | 16-33 | 24.6 |
| L-Histidine | 2-20 | 7-14 | 10.3 |
| L-Methionine | 0.1-5 | 1.4-2.8 | 2.1 |
| L-Phenylalanine + | 5-50 | 23‥47 | 35.3 |
| L-Lysine | | | |
| L-Threonine + | 5-50 | 32-64 | 48.3 |
| L-Arginine | | | |
| L-Tyrosine | 1-20 | 5-11 | 7.9 |
| Na⁺, mM | 135-150 | 137-147 | 147 |
| K⁺, mM | 2.5-4.0 | 2.7-3.9 | 2.9 |
| Cl⁻, mM | 110-135 | 116-135 | 130 |
| Ca⁺², mM | 1.0-1.6 | 1.0-1.5 | 1.15 |
| Mg⁺², mM | 0.8-1.6 | 1.0-1.5 | 1.12 |
| Glucose (dextrose), | 10-150 | 30-100 | 94 |
| mg/dL | | | |

In some embodiments, the cerebrospinal perfusion fluid is simplified further, such as according to the following:

| | | More | Still More |
|---|---|---|---|
| | Preferred | Preferred | Preferred Range |
| Component | Range | Range | or Amount |
| Albumin, g/dL, | 0.05-2.0 | 1.5-1.9 | 1.67 |
| Na⁺, mM | 135-150 | 137-147 | 147 |
| K⁺, mM | 2.5-4.0 | 2.7-3.9 | 2.9 |
| Cl⁻, mM | 110-135 | 116-135 | 130 |
| Ca⁺², mM | 1.0-1.6 | 1.0-1.5 | 1.15 |
| Mg⁺², mM | 0.8-1.6 | 1.0-1.5 | 1.12 |
| Glucose (dextrose), | 10-150 | 30-100 | 94 |
| mg/dL | | | |

For example, the poly-fluorinated, oxygen-carrying compound can be omitted. Or, the amino acid nutrient components can be omitted. Ions are maintained to the degree required to avoid damage to cerebrospinal tissue. Appropriate amounts of oncotic agents are preferred. The cerebrospinal perfusion fluid preferably contains one or both of the lipid and albumin components in the amounts recited above. The lipid can be derived from a lipid source containing lipids of a type used to form biological membranes. Preferably, the lipids are phospholipids, such as the phospholipid, predominately lecithin, isolated from egg yolk. Where lipids are present, the cerebrospinal perfusion fluid is typically emulsified.

Generally, tissues and cells will not fare well if exposed to large volumes of non-physiologic ionic solutions. Accordingly, appropriate electrolyte compositions at the tissue level are important when it is considered that the circulatory method of the present invention could dilute of electrolytes from the region, to the detriment of cell membrane function. Desirably, sodium, potassium, calcium, magnesium, and chloride ions are carefully balanced in the antimicrobial formulations of the present invention to create, to the degree possible, normal extra-cellular compositions.

The formulations of the invention preferably exclude four amino acids, glutathione, cysteine, ornithine and glutamine, from the group of amino acids included in the formulation, and preferably include sodium bicarbonate in an amount sufficient to increase the buffering capacity of the nutrient solution, in order to more closely resemble cerebrospinal fluid of the subject.

Kits for conveniently and safely generating fluorocarbon nutrient emulsion or a corresponding vehicle lacking poly-fluorinated, oxygen-carrying compound are described for example in U.S. patent application Ser. No. 09/619,414, filed Jul. 19, 2000 (the specific formulations and kits described therein are incorporated by reference as outlined below).

Methodology

In accordance with a preferred method of the present invention, the transforming composition is circulated through this cerebrospinal fluid route by injecting it into brain vesicles and withdrawing it from the cisterna magna or the spinal subarachnoid space to nourish and to treat central nervous tissues. In other instances the fluid can be injected into the subarachnoid space and withdrawn from another subarachnoid position.

The cerebrospinal perfusion fluid comprising a gene therapy agent can be introduced into the subarachnoid spaces through a catheter that transverses the skull or spinal column and the meninges. The delivery point can be the lateral ventricles, subarachnoid space around the brain, cisterna magna or anywhere along the spine. The cerebrospinal perfusion fluid can be withdrawn from the subarachnoid space from any of these locations using a similar catheter. The cerebrospinal perfusion fluid can be returned to the delivery system, reconditioned as necessary to add components that have been consumed or remove undesirable components that have accumulated, and then returned to the subarachnoid space in recirculating fashion. This process can be continued for days if necessary, thereby directly exposing the neuraxis to a gene therapy agent or transformed cells over an extended period of time.

This method has several advantages over other routes of administration, such as direct exposure of the nervous system tissue to the gene therapy agent by a simple bolus injection of the agent or cells into the subarachnoid space. This invention provides a method of circulating the gene therapy agent throughout the neuraxis, this exposing nervous system tissue to the agent or cells much more uniformly than would otherwise be possible. It also provides a method of maintaining the gene therapy agent within a narrow concentration range, avoiding the necessity of high concentrations over time. According to this method, the nervous system tissue can be exposed to the agent or cells for extended period time, such as days, if necessary. Further, this method minimizes the amount of agent or cells necessary to achieve a therapeutic effect.

It is preferable to establish a flow pathway from the entry catheter (e.g., a ventricular catheter into a lateral ventricle of the brain) to an exit point at a different location in the cerebral spinal pathway (e.g., into the intrathecal space of the lumbar (such as L4-L5) region of the spine) without prematurely inserting a CSF containing, for example, transforming nucleic acid, oxygen-carrying compound, other emulsified components, or the like.

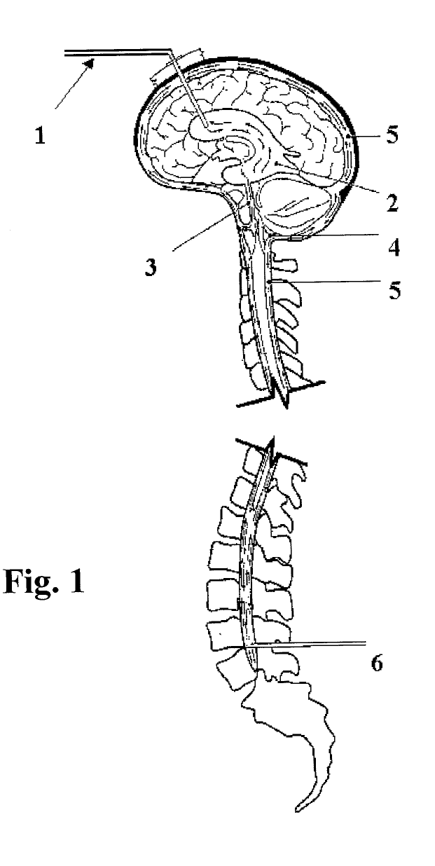

As illustrated in FIG. 1, a ventricular catheter 1 is inserted into a lateral ventrical 2. Via aqueduct 3, cisterna magna 4 and subarachnoid spaces 5, a flow pathway can be established to a lumbar outflow catheter 6. When the inflow and outflow catheters are established (typically with suitable controls to monitor intracranial and intraspinal pressure), the vehicle can be used to establish the existence of a flow pathway (such as that illustrated) from the inflow catheter to the outflow catheter. Preferably, the vehicle is infused under gravity feed, with the pressure head designed to avoid excessive intracranial pressure. Once established, the vehicle can be substituted with the cerebrospinal perfusion fluid.

It will be apparent that more than two catheters can be used, though additional catheters are not preferred. Care is taken to monitor the intracranial pressure to assure that flow rates do not cause excessive pressure.

Cerebrospinal perfusion fluid is preferably perfused through the cerebrospinal pathway for a period of time or perfusion volume adapted to effectively presents the gene therapy agent (e.g. nucleic acid or transformed cell). The volume perfused is, in one embodiment, preferably about 15 CSF volumes, where a “CSF volume” is the average volume of CSF fluid found in animals of comparable age to the subject. Preferably, at least about 1, 2, 4, 8 or 30 CSF volumes are used. In adult humans, for example, a flow rate in the range of 300-3,600 mL/hr is expected, resulting in the exchange of about 2-22 CSF volumes/hr. In human adults, the perfusion is preferably with 300 to 3,600 mL/hr.

The perfusion can be conducted, for example, for 6, 12, 24 or 48 or more hours. Preferably the perfusion is conducted for between 6 hours and 48 hours or more preferably between 12 hours and 24 hours. More preferably, the perfusion is conducted for at least about 24 hours; and preferably the perfusion is conducted for no more than about 120 hours (and in one embodiment, no more than about 72 hours).

Preferred treatment subjects among animals are mammals, preferably humans.

Oxygen-Carrying Compounds

Generally, the preferred compounds for use as non-aqueous oxygen transfer components are fluorocarbons, such as perfluorocarbons, perfluorinated alkyl polyethers, fluoroethers, fluoramines, etc. While compounds within these groups range in gram molecular weight from 250 to 7000 g/mole, their selection for use as non-aqueous transport components are based upon the combination of features of the proper vapor pressure, molecular weight, viscosity, ability to form emulsions, emulsion stability and tissue distribution. Not only do fluorocarbons possess appropriate properties but they are for the most part non-toxic. One chief advantage of the CSF circulation route is that most or all of the formulation can be removed by flushing the subarachnoid space with vehicle at the time of treatment termination. In this way long term cellular retention of oxygenating liquids can be avoided.

Poly-fluorinated, oxygen-carrying compounds are known in the art. The basic requirement is effectiveness in carrying physiologically useful amounts of oxygen. Factors involved in selecting preferred such compounds include oxygen capacity, tissue retention (preferably minimized), emulsion stability, toxicity, and the like. Such compounds are described in numerous publications (for example, in: Riess et al., “Design Synthesis and Evaluation of Fluorocarbons and Surfactants for In vivo Applications New Perfluoroalkylated Polyhydroxylated Surfactants”, Biomat. Artif. Cells Artif. Organs, 16:421-430 (1988); Riess, Reassessment of criteria for the Selection of Perfluorochemicals for Second-Generation Blood Substitutes: Analysis of Structure/Property Relationships, Artificial Organs 8:44-56 (1984); Riess, et al., Design, Synthesis and Evaluation of Fluorocarbons and Surfactants for In Vivo Applications New Perfluoroalkylated Polyhydroxylated Surfactants, Biomat. Artif. Cells Artif. Organs 16:421-430 (1988); Riess, et al., Solubility and Transport Phenomena in Perfluorochemicals Relevant to Blood Substitution and Other Biomedical Applications, Pure & Applied Chem., 54:2383-2406 (1982); Yamanouchi, et al., Quantitative Structure-In Vivo Half-Life Relationships of Perfluorochemicals for Use as Oxygen Transporters, Chem., Pharm. Bull., 33:1221-1231 (1985); Lowe, et al., Perfluorochemicals: Blood Substitutes and Beyond Adv. Mater, 3:87-93 (Feb., 1991); Riess, et al., Fluorocarbon-Based In Vivo Oxygen Transport and Delivery Systems Vox Sang, 61:225-239 (December 1991); and Weers, et al., U.S. Pat. No. 5,914,352).

Among preferred poly-fluorinated, oxygen-carrying compounds are those of the formula

CₘFₘ₊₁—CH═CH—CₙFₙ₊₁,

where m and n or independently at least 1 and m+n equals 6 to 10. Preferably, the double bond is trans. One preferred poly-fluorinated, oxygen-carrying compound is trans-Bis-perfluorobutyl ethylene (m and n each equal 4), which is also known as F44E. F44E formulations have a 25% greater oxygen carrying capacity than that of a prior nutrient solution made with perfluorodecalin. Bell et al., Neurology 37: 133, 1987. Formulations comprising F44E are less viscous and relatively easier to perfuse.

Also preferred are those of the formula

CₘFₘ₊₁—O—CₙFₙ₊₁,

where m and n or independently at least 1 and m+n are equals 6 to 9 (or 8). One of the perfluoro alkyls can be substituted with a halo from Br (preferably), Cl or I. Further preferred are those of the formula

CₘFₘ₊₁—R

where m is 8 (or 10) to 12 and R is Br, Cl, I, or C₁-C₃ alkyl.

Besides fluorocarbon based products, cell-free hemoglobin and liposome encapsulated hemoglobin may also be used as artificial oxygen carriers. Hemoglobin is a 4 subunit protein that is the naturally occurring oxygen carrier in red blood cells. Cell-free hemoglobin rapidly dissociates in the bloodstream, so artificial hemoglobins are chemically modified to prevent breakdown. Artificial hemoglobins can be the product of surface modification, crosslinking, or polymerization. The production and use of cell-free hemoglobin is detailed in a number of publications (for example, U.S. Pat. Nos. 5,438,041; 5,770,727; 5,952,470; 5,691,453; 5,618,919; 5,599,907; 5,739,011; 5,563,254; 5,449,759; 5,128,452; 5,827,693, and 5,312,808). Hemoglobin can also be prevented from degradation by being encapsulated within a protective barrier, as in the case with liposome encapsulated hemoglobin, the production and use of which is presented in a number of publications (for example, U.S. Pat. Nos. 5,049,391; 4,133,874; 4,776,991; 4,425,334, and 4,532,130).

### EXAMPLE 1

### Treatment of Amyotrophic Lateral Sclerosis

A baculovirus-dervived vector (see, Sarkis et al, Proc. Natl. Acad. Sci. US, Vol. 97(26): 14638-14643, 2000), encoding superoxide dismutase 1 (SOD1), is added to a perfusion formulation such as:

| Component | Quantity/2138 mL | |
|---|---|---|
| NaCl, USP | 15.3 | g |
| NaHCO₃, USP | 4.14 | g |
| KCl, USP | 0.46 | g |
| MgCl₂—6H₂O, USP | 0.48 | g |
| CaCl₂—2H₂O, USP | 0.36 | g |
| Dextrose, USP | 2 | g |
| Albumin (Human), USP (20%) | 40 | g |
| L-lysine HCl, USP | 6.48 | mg |
| L-alanine, USP | 6.86 | mg |
| L-serine, USP | 5.90 | mg |
| L-threonine, USP | 7.10 | mg |
| L-arginine, USP | 4.48 | mg |
| L-leucine, USP | 2.96 | mg |
| L-isoleucine, USP | 1.24 | mg |
| L-valine, USP | 3.98 | mg |
| L-phenylalanine, USP | 1.98 | mg |
| L-tyrosine, USP | 1.90 | mg |
| L-histidine, USP | 2.46 | mg |
| L-methionine, USP | 0.50 | mg |
| NaH₂PO₄, USP | 8.20 | mg |
| Na₂HPO₄, USP | 1.22 | mg |
| α-ketoglutaric acid | 60 | mg |
| Water for Injection, USP | 2056 | mL |

This formulation is then perfused through a human central nervous system via ventriculo-lumbar perfusion for a period of 48 hours, thus transforming neurons to produce SOD1.

### EXAMPLE 2

### Treatment of Amyotrophic Lateral Sclerosis

Other recombinant adenoviruses, adeno associated viruses, herpes viruses and lenti viruses are used to encode neuroprotective proteins calbindin, the anti-apoptotic proto-oncogene bcl-2 (see, e.g., Wei et al., J Neurochem 75(1):81-90, 2000), glial-derived neurotrophic growth factors (NGF, BDNF and NT3), epidermal growth factor (EGF), fibroblast growth factors (FGF 1-9), insulin (IGF-1), platelet-derived growth factors (PDGF—A, B &C), vascular endothelial growth factor (VEGF), and ciliary neurotrophic factor (CNTF). (See, Alisk & Davidson, Human Gene Therapy, 11(17): 2315-2329, 2000).

### EXAMPLE 3

### Treatment of Obesity

An adenovirus vector expressing a cDNA encoding the protein leptin, (see, Muzzin et al, Regulatory Peptides, 92(1-3 Special Issue SI): 57-64, 2000), is added to a perfusion formulation as follows:

| Component | Quantity/2138 mL | |
|---|---|---|
| NaCl, USP | 15.3 | g |
| NaHCO₃, USP | 4.14 | g |
| KCl, USP | 0.46 | g |
| MgCl₂—6H₂O, USP | 0.48 | g |
| CaCl₂—2H₂O, USP | 0.36 | g |
| Dextrose, USP | 2 | g |
| Albumin (Human), USP (20%) | 40 | g |
| L-lysine HCl, USP | 6.48 | mg |
| L-alanine, USP | 6.86 | mg |
| L-serine, USP | 5.90 | mg |
| L-threonine, USP | 7.10 | mg |
| L-arginine, USP | 4.48 | mg |
| L-leucine, USP | 2.96 | mg |
| L-isoleucine, USP | 1.24 | mg |
| L-valine, USP | 3.98 | mg |
| L-phenylalanine, USP | 1.98 | mg |
| L-tyrosine, USP | 1.90 | mg |
| L-histidine, USP | 2.46 | mg |
| L-methionine, USP | 0.50 | mg |
| NaH₂PO₄, USP | 8.20 | mg |
| Na₂HPO₄, USP | 1.22 | mg |
| α-ketoglutaric acid | 60 | mg |
| Water for Injection, USP | 2056 | mL |

This formulation is then perfused through a human central nervous system via ventriculo-lumbar perfusion for a period of 48 hours, thus transforming neurons to produce the satiety factor leptin.

Definitions

The following terms shall have, for the purposes of this application, the respective meanings set forth below.

antisense molecule. An “antisense molecule” is a molecule adapted to selectively bind to a nucleic acid in a cell to disrupt the target nucleic acid's function. These are typically “oligonucleotides,” but those of ordinary skill will recognize that the ordinary use of “antisense oligonucleotide” has come to refer to a variety of molecules based on standard nucleic acids but having various modifications and linkages, as now widely understood in the art. Thus, the term “antisense molecule” includes the ordinary meanings in the art of “antisense oligonucleotide.”

gene therapy. As used herein, “gene therapy” includes any intervention in an animal (preferably a mammal, more preferably a human) that (i) causes a cell in the animal to express (as RNA or protein) a recombinant nucleic acid, whether such expression is transient or stable, (ii) causes a change in the cell's genome, such as an insertion, that changes the cell's pattern of gene expression, (iii) provides a transformed cell to the animal, or (iv) alters the expression of a gene in the cell. Hence, gene therapy includes transformations with anti-sense constructs and uses of nucleic acid-based vaccines.

gene therapy agent. A nucleic acid for gene therapy can be an antisense molecule or a transforming nucleic acid, including a transforming nucleic acid adapted to direct the production of an antisense molecule. A transforming nucleic acid can transform by positively encoding a gene product, inserting a promoter (such as in an appropriate gene trap such as described in U.S. Pat. No. 6,080,576), disrupting a gene or gene expression, or the like.

nutrient-providing effective amount. A nutrient-providing effective amount of a substance is an amount that can be expected, provided sufficient amounts of other nutrients, to increase metabolism or reproduction of mammalian cells compared with nutrient solutions lacking that substance.

oncotic agent. By oncotic agent is meant substances, generally macromolecules, that are of a size that is not readily able to leave the body cavity or other fluid containing body spaces (such as the cerebrospinal pathway, including the cerebral ventricles and subarachnoid spaces) into which they are inserted. Such oncotic agents are exemplified by blood plasma expanders which are known in general as macromolecules having a size sufficient to inhibit their escape from the blood plasma through the circulatory capillary bed into the interstitial spaces of the body. Serum albumin, preferably human serum albumin, is one well known blood plasma protein that can be used as an oncotic agent. Polysaccharide blood plasma expanders are often glucan polymers. For example, Hetastarch (a product of American Home Products) is an artificial colloid derived from a waxy starch composed almost entirely of amylopectin with hydroxyethyl ether groups introduced into the alpha (1-4) linked glucose units. The colloid properties of a 6% solution (wt/wt) of hetastarch approximate those of human serum albumin. Other polysaccharide derivatives may be suitable as oncotic agents in the blood substitute according to the invention. Among such other polysaccharide derivatives are hydroxymethyl alpha (1-4) or (1-6) polymers and cyclodextrins. In general, it is preferred that the polysaccharide is one that is non-antigenic. High molecular weight agents such as Dextran 70 having a molecular weight of about 70,000 Daltons are generally less preferred because they increase viscosity of the colloidal solution and impair the achievement of high flow rates. Preferably, the oncotic agent is in an amount effective to provide, in conjunction with other components of a fluorocarbon nutrient emulsion or a nutrient solution, an oncotic pressure of one to seven torr.

or. The conjunction “or” is used to express that at least one of the recited alternatives linked by or is applicable in a given context and to include the conjunctive sense, joining two or more of the recited alternatives. In other words, unless the context indicates a contrary meaning, “or” includes the meaning sometimes expressed as “and/or.”

polynucleotide or nucleic acid. The terms polynucleotide(s) or nucleic acid(s) (herein “polynucleotide(s)”) generally refer to any polyribonucleotide or polydeoxyribonucleotide, which can be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that can be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

respiration. Respiration is the physical and chemical processes by which an organism supplies its cells and tissues with the oxygen needed for metabolism and, preferably, relieves them of the carbon dioxide formed in energy-producing reactions.

respiration-supporting amount. A respiration-supporting amount of oxygen is an amount that would, in model experiments, provide a statistically significant reduction in morbidity following a focal ischemic event.

transformed cell. A cell is transformed if a recombinant nucleic acid is introduced into it or its ancestor so as to temporarily or stably (1) cause the cell to express a polypeptide or RNA in an amount not otherwise expressed by the cell or (2) interfere with the translation or transcription of a nucleic acid normally found in the cell.

transforming composition. A transforming composition is a composition containing a gene therapy effective amount of transformed cells or gene therapy agent.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references. Particular attention is given to cited text describing vectors and methods of their use, transformation-enhancing methods and compositions, and antisense molecules and their use.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of delivering to neurological tissue a transforming composition or a transformed cell, the method comprising:
a. injecting a physiologically acceptable cerebrospinal perfusion fluid into a first catheter into the cerebrospinal pathway, which cerebrospinal perfusion fluid has a gene therapy effective amount of gene therapy agent or transformed cells;
b. withdrawing fluid at a second catheter in the cerebrospinal pathway to create a flow and flow pathway between the first and second catheters; and
c. maintaining the flow for a period of time adapted to perfuse at least 1 CSF volume.

2. The method of claim 1, wherein the flow is maintained for between 6 hours and 48 hours.

3. The method of claim 1, wherein fluid is adapted to not carry a respiration-supporting amount of oxygen.

4. The method of claim 1, wherein the gene therapy agent comprises a nucleic acid adapted to be expressed in the target cell or to be incorporated into the genome of the target cell to alter gene expression.

5. The method of claim 1, wherein transformed cells are delivered.

6. The method of claim 1, wherein an antisense molecule is delivered.

7. The method of claim 1, comprising maintaining the flow for a period of time adapted to perfuse at least 2 CSF volumes.

8. The method of claim 1, comprising maintaining the flow for a period of time adapted to perfuse at least 4 CSF volumes.

9. The method of claim 1, comprising maintaining the flow for a period of time adapted to perfuse at least 8 CSF volumes.

10. The method of claim 1, comprising maintaining the flow for a period of time adapted to perfuse at least 15 CSF volumes.

11. A method of delivering to neurological tissue a transforming composition or a transformed cell, the method comprising:
a. injecting a physiologically acceptable cerebrospinal flushing fluid into a first catheter into the cerebrospinal pathway, which cerebrospinal flushing fluid has an effective amount a gene therapy agent effective to transform neural cells to express calbindin, bal-2, leptin, superoxide dismutase, a glial-derived neurotrophic growth factor which is NGF, BDNF or NT3, epidermal growth factor, a fibroblast growth factor which is FGF 1-9, insulin-like growth factor-1, a platelet-derived growth factor which is PDGF—A, B or C, vascular endothelial growth factor, or ciliary neurotrophic factor, or transformed cells;
b. withdrawing fluid at a second catheter in the cerebrospinal pathway to create a flow and flow pathway between the first and second catheters; and
c. maintaining the flow for a period of time adapted to flush at least 1 CSF volume.

12. The method of claim 11, wherein the gene therapy agent expresses calbindin, bal-2, leptin, superoxide dismutase, a glial-derived neurotrophic growth factor which is NGF, BDNF or NT3, epidermal growth factor, a fibroblast growth factor which is FGF 1-9, insulin-like growth factor-1, a platelet-derived growth factor which is PDGF—A, B or C, vascular endothelial growth factor, or ciliary neurotrophic factor.

13. The method of claim 12, wherein fluid comprises:

| Component | Range |
|---|---|
| α-Ketoglutaric Acid, μg/mL | 5-40 |
| Amino Acids, μg/mL | |
| L-Isoleucine + L-Leucine | 5-50 |
| L-Valine | 5-50 |
| L-Alanine | 5-50 |
| L-Serine | 5-50 |
| L-Histidine | 2-20 |
| L-Methionine | 0.1-5 |
| L-Phenylalanine + L-Lysine | 5-50 |
| L-Threonine + L-Arginine | 5-50 |
| L-Tyrosine | 1-20 |
| Na⁺, mM | 135-150 |
| K⁺, mM | 2.5-4.0 |
| Cl⁻, mM | 110-135 |
| Ca⁺², mM | 1.0-1.6 |
| Mg⁺², mM | 0.8-1.6 |
| Glucose (dextrose), mg/dL | 10-150 |

14. The method of claim 13, wherein fluid further comprises:

| Component | Range |
|---|---|
| Albumin, g/dL, | 0.05-2.0 |

15. A method of delivering to neurological tissue a transforming composition or a transformed cell, the method comprising:
a. injecting a cerebrospinal perfusion fluid into a first catheter into the cerebrospinal pathway, which cerebrospinal perfusion fluid has a gene therapy effective amount of transforming nucleic acid, wherein the cerebrospinal flushing fluid further comprises an emulsion-forming effective amount of a lipid composition comprised of lipids found in biological membranes;
b. withdrawing fluid at a second catheter in the cerebrospinal pathway to create a flow and flow pathway between the first and second catheters; and
c. maintaining the flow for a period of time adapted to flush at least 1 CSF volume.

16. The method of claim 15, wherein the lipids are phospholipids.
